(19) United States
(12) Patent Application Publication
(10) Pub. No.: US 2016/0296246 A1
(43) Pub. Date: Oct. 13, 2016

(54) FORCEPS WITH METAL AND POLYMERIC ARMS

(71) Applicant: Novartis AG, Basel (CH)

(72) Inventor: Philipp Schaller, Rhein (CH)

(21) Appl. No.: 14/684,937

(22) Filed: Apr. 13, 2015

(51) Int. Cl. A61B 17/30; A61F 9/007

(52) U.S. Cl. CPC A61B 17/30; A61F 9/007; A61B 2017/305; A61B 2017/00526

(57) ABSTRACT

An instrument includes a shaft comprising a lumen and a first arm extending from the lumen. The first arm includes a first proximal portion formed of a metal first material. The first arm also includes a first distal portion having a first tip, the first distal portion formed of a second material. The instrument also includes a second arm extending from the lumen. The second arm includes a second proximal portion formed of the metal first material. The second arm also includes a second distal portion having a second tip, the second distal portion formed of the second material. The first and second shaft engaging portions are configured to engage the shaft such that movement of the shaft with respect to the first and second arms moves the first and second arms together such that the first tip moves towards the second tip in a manner permitting grasping of tissue.

### FIELD OF THE INVENTION

The present disclosure relates to apparatuses and methods for ophthalmic medical procedures, and more particularly, to apparatuses and methods involving surgical instruments for such procedures.

### BACKGROUND

Many microsurgical procedures require precision cutting and/or removal of various body tissues. For example, Internal Limiting Membrane (ILM) removal and epi-retinal membrane (ERM) removal are microsurgical procedures for treating macular surface diseases. These types of microsurgical procedures require skill and patience. To be most effective, precisely and carefully constructed surgical instruments are used for each segment of the microsurgical procedure.

The microsurgical procedure itself includes grasping an edge of an ILM or ERM membrane, and peeling the membrane. The technique itself is a two-step procedure. First, the surgeon gains an edge of the ILM or ERM membrane. This is the process for rendering the membrane in a manner suitable for grasping. Some surgeons use for example a scraper or a forceps to gain the membrane edge. Next, the surgeon introduces a special forceps to grasp and peel the membrane. However, since each step requires such precision to avoid damaging surrounding tissue, such as the retina, a surgeon may sometimes scrape and then attempt to grasp the tissue multiple times during a single surgical procedure.

There is a need for continued improvement in the use and operability of surgical systems and tools for various ophthalmic procedures. The systems and methods discussed herein are arranged to address one or more of the deficiencies in the prior art.

### SUMMARY

According to one example, an ophthalmic surgical instrument includes a shaft comprising a lumen having a longitudinal axis and a first arm extending from the lumen. The first arm includes a first proximal portion formed of a metal first material, the first proximal portion comprising a first shaft engaging portion that is angled away from the longitudinal axis of the shaft and a first distal portion attached onto the first proximal portion and having a first tip, the first distal portion formed of a second material different than the metal first material. The instrument further includes a second arm extending from the lumen. The second arm includes a second proximal portion formed of the metal first material, the second proximal portion comprising a second shaft engaging portion that is angled away from the longitudinal axis of the shaft, and a second distal portion attached onto the second proximal portion and having a second tip, the second distal portion formed of the second material. The first and second shaft engaging portions are configured to engage the shaft such that movement of the shaft with respect to the first and second arms moves the first and second arms together such that the first tip moves towards the second tip in a manner permitting grasping of tissue.

An ophthalmic instrument includes a shaft comprising a lumen. The instrument also includes a first arm extending from the lumen. The first arm includes a first proximal portion formed of a metal material, the first proximal portion comprising a first sized portion and a second sized portion extending distally from the first sized portion, the second sized portion being smaller in at least one dimension than the first sized portion. The first arm also includes a first distal portion formed over the second sized portion of the first proximal portion, the first distal portion comprising a polymeric material. The instrument also includes a second arm extending from the lumen. The second arm includes a second proximal portion formed of the metal material, the second proximal portion comprising a first sized portion and a second sized portion extending distally from the first sized portion, the second sized portion being smaller in at least one dimension than the first sized portion. The second arm also includes a second distal portion formed over the second sized portion of the second proximal portion, the second distal portion comprising the polymeric material.

A method for fabricating ophthalmic instruments includes forming a plurality of metal forceps. Each of the metal forceps includes a first arm having a first extension that extends distally from the first arm and is smaller than the first arm along at least one dimension and a second arm having a second extension that extends distally from the second arm and is smaller than the second arm along at least one dimension. The method further includes molding a first type of polymeric tip over the first extension and the second extension of a first one of the plurality of metal forceps. The method further includes molding a second type of polymeric tip over the first extension and the second extension of a second one of the plurality of metal forceps.

### BRIEF DESCRIPTION OF THE DRAWINGS

The accompanying drawings illustrate embodiments of the devices and methods disclosed herein and together with the description, serve to explain the principles of the present disclosure.

FIGS. 1A and 1B are diagrams showing an illustrative forceps made of metal proximal portions and polymeric distal portions according to one example incorporating the principles described herein.

FIGS. 2A and 2B are diagrams showing a perspective view of a forceps before and after the distal portions are formed on a metal skeleton according to one example incorporating the principles described herein.

FIG. 3 is a diagram showing a cross-sectional view of an interface between a metal proximal portion and a polymeric distal portion of a forceps according to one example incorporating the principles described herein.

FIGS. 4A, 4B, and 4C are diagrams showing illustrative forceps with similar metal proximal portions and varying polymeric distal portions according to various examples incorporating the principles described herein.

FIGS. 5A and 5B are diagrams showing illustrative forceps with similar metal proximal portions and varying polymeric distal portions according to various examples incorporating the principles described herein.

FIG. 6 is a flowchart showing an illustrative method for fabricating a forceps with metal proximal portions and polymeric distal portions according to one example incorporating the principles described herein.

### DETAILED DESCRIPTION

For the purposes of promoting an understanding of the principles of the present disclosure, reference will now be made to the embodiments illustrated in the drawings, and specific language will be used to describe the same. It will nevertheless be understood that no limitation of the scope of the disclosure is intended. Any alterations and further modifications to the described devices, instruments, materials, methods, and any further application of the principles of the present disclosure are fully contemplated as would normally occur to one skilled in the art to which the disclosure relates. In particular, it is fully contemplated that the features, components, and/or steps described with respect to one embodiment may be combined with the features, components, and/or steps described with respect to other embodiments of the present disclosure. For simplicity, in some instances the same reference numbers are used throughout the drawings to refer to the same or like parts.

The present disclosure relates to a forceps with arms that have a metal proximal portion and a distal polymeric portion. Such a forceps is usable in ophthalmic surgical procedures and associated methods. One example of a forceps used in an ILM procedure is a tool that includes a shaft with a lumen formed coaxially through the center. In some examples, the shaft is less than one millimeter in diameter. Such a small size allows the instrument to penetrate and treat even small organs, such as eyes. Extending from the shaft are two small, outwardly biased arms. The arms are axially repositionable with respect to the shaft such that moving the shaft in a distal direction with respect to the arms slides the shaft over the arms and pushes the arms together to provide a grasping force.

A conventional forceps may be made entirely of a metal material or entirely of a polymeric material. Each type of forceps provides a number of advantages as well as a number of drawbacks. For example, use of a metal forceps provides a stronger grasping force as sliding the shaft over the outwardly biased arms forces the arms together. But, the tips of a metal forceps are difficult to form into specific shapes, particularly considering the small size of the metal arms. Polymeric forceps, on the other hand, allow for finer precision when fabricating the tips. But, polymeric forceps do not provide as strong of a grasping force.

According to principles described herein, a forceps includes two arms extending from the lumen of a shaft. Each arm has a proximal portion that is formed of metal or other material with the desired properties. In one example, each arm also includes a distal portion that is made of a polymeric material or other material that is suitable for use in an injection molding or three-dimensional (3D) printing process. In one example, the polymeric material is injection molded at the distal end of the proximal portions of the arm. As will be described in further detail below, such a forceps provides the strength and grasping force of metal forceps while the polymeric distal portions allow for cost effective production of a variety of defined tips that can be suited for various purposes. While the following discussion describes the use of polymeric distal portions, it is understood that the polymeric distal portions may be made of another material besides polymer. For example, the distal portions may be made of a metal material that is suitable for injection molding or 3D printing.

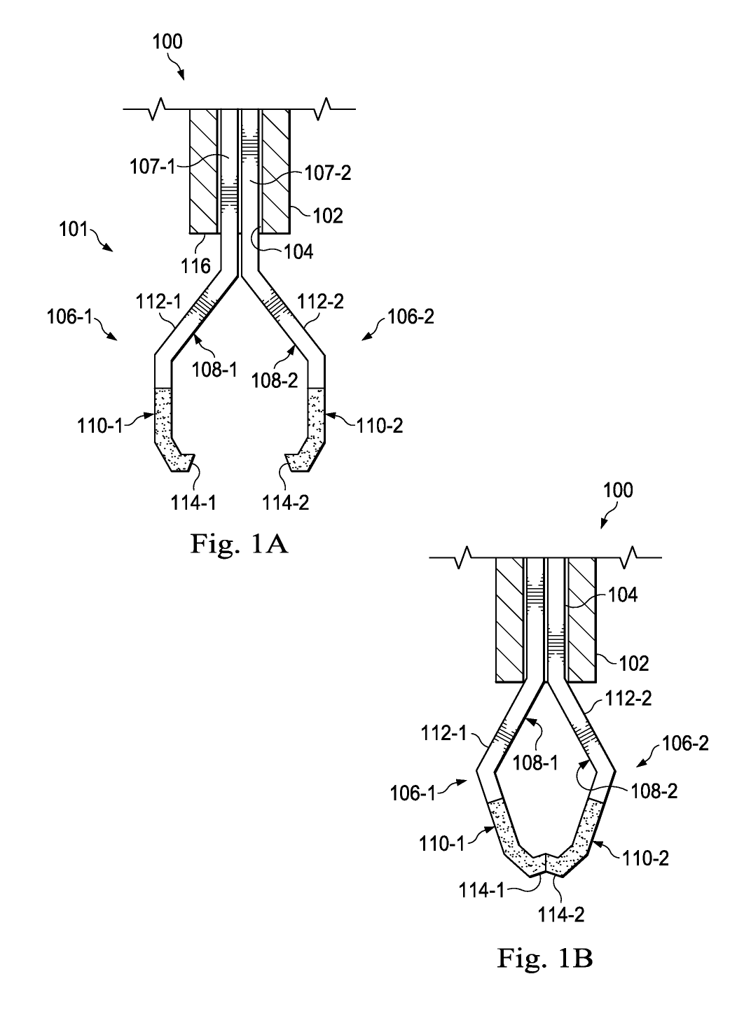

FIGS. 1A and 1B are diagrams showing an illustrative forceps 100 with arms 106-1, 106-2 having a metal proximal portion 108-1, 108-2 and a polymeric distal portion 110-1, 110-2. FIG. 1A illustrates the forceps 100 in an expanded position. FIG. 1B illustrates the forceps 100 in a closed position. According to the present example, the forceps 100 includes a shaft 102 with a lumen 104 coaxially extending therethrough.

The shaft 102 may be made of a metal material. Thus, the shaft 102 may be substantially rigid. The shaft 102 may be designed to penetrate the eye, or to be inserted into a small cannula that has penetrated the eye, so that the forceps which can be extended from the shaft 102 can reach the surgical site within the eye.

The first arm 106-1 and the second arm 106-2 extend from the lumen 104, together forming the jaws 101 of the forceps 100. The arms 106-1, 106-2 respectively extend from bodies 107-1, 107-2 in the lumen 104. The bodies 107-1, 107-2 may be axially actuated to open and close the jaws 101 of the forceps 100. The first arm 106-1 includes a proximal portion 108-1 and a distal portion 110-1 having an inwardly facing first tip 114-1. Likewise, the second arm 106-2 includes a proximal portion 108-2 and a distal portion 110-2 having an inwardly facing second tip 114-2. The first tip 114-1 faces the second arm 106-2 and the second tip 114-2 faces the first arm 106-1.

In some examples, the arms 106-1, 106-2 may be relatively small in size. For example, in some embodiments, the arms 106-1, 106-2 have a width within a range of 0.05 to 0.3 millimeters. The arms 106-1, 106-2 may extend from the shaft 102 as far as 1 to 3 millimeters. Other sizes, both larger and smaller, are contemplated as well.

In one example, the forceps 100 are biased to the open condition. The forceps 100 are closed to grasp tissue or other elements by moving the shaft 102 in an axial direction relative to the bodies 107-1, 107-2 and arms 106-1, 106-2. As the shaft 102 moves toward the distal ends of the arms 106-1, 106-2, a leading edge 116 of the shaft 102 abuts against the separated proximal portions 108-1, 108-2, and forces the two arms 106-1, 106-2 towards each other. Specifically, the shaft 102 may act as a sleeve that presses the arms 106-1, 106-2 together as it moves to cover a portion of the arms 106-1, 106-2. The arms 106-1, 106-2 may be biased to automatically expand apart as they extend further from the shaft 102.

The proximal portions 108-1, 108-2 of the arms 106-1, 106-2 are formed of a metal material, and may thus be referred to as metal portions, metal arms, or metal proximal portions. Various metal materials, such as stainless steel, may be selected for use to form the proximal portions 108-1, 108-2. Other metals that provide the desired stiffness and durability may be used as well. In some examples, the proximal portions 108-1, 108-2 of the arms 106-1, 106-2 are made of the same material as the bodies 107-1, 107-2, and may form a monolithic element of the forceps 100.

The proximal portions 108-1, 108-2 also include shaft engaging portions 112-1, 112-2. Specifically, the first proximal portion 108-1 includes a first shaft engaging portion 112-1 and the second proximal portion 108-2 includes a second shaft engaging portion 112-2. In the embodiment of FIGS. 1A and 1B, the shaft engaging portions 112-1, 112-2 form the outer side of the arms 106-1, 106-2. Because the arms 106-1, 106-2 are formed or shaped to be outwardly biased, when the shaft 102 is not positioned or engaged with the shaft engaging portions 112-1, 112-2 of the arms 106-1, 106-2, they are naturally disposed in an open or separated position. As the shaft 102 disengages the shaft engaging portions 112-1, 112-2, the arms 106-1, 106-2 tend to move in an outward direction so as to cause the tips 114-1, 114-2 to move away from each other. The amount of outward bias is sufficient to allow the arms 106-1, 106-2 to open when the shaft 102 is retracted but not so strong so as to make it too difficult to slide the shaft 102 over a part of the arms 106-1, 106-2. When the shaft 102 is moved over a part of the arms 106-1, 106-2, as illustrated in FIG. 1B, the shaft engaging portions 112-1, 112-2 press against the leading edge 116 or inner surface of the shaft 102 causing the arms 106-1, 106-2 to elastically deflect or flex from the open position in FIG. 1A to the closed position in FIG. 1B. Because the shaft engaging portions 112-1, 112-2 of the proximal portions 108-1, 108-2 are angled away from the longitudinal axis of the shaft 102, a desired grasping force can be applied between the tips 114-1, 114-2 when the shaft 102 is slid over a part of the arms 106-1, 106-2.

The distal portions 110-1, 110-2 form the tissue contacting element of the forceps 100 and are made of a polymeric material. The distal portions are formed in a different fabrication process than the process used to form the proximal portions 108-1, 108-2. Thus, the distal portions 110-1, 110-2 are attached to the proximal portions 108-1, 108-2. The distal portions 110-1, 110-2 are formed of a material that is different than the material used to form the metal proximal portions 108-1, 108-2. For example, the distal portions 110-1, 110-2 may be formed of a polymer material. Thus, the distal portions 110-1, 110-2 may be referred to as polymeric portions or polymeric distal portions. In some examples, however, other materials such as metals that are suited to an injection molding process or 3D printing process may be used to form the distal portions 110-1, 110-2.

In preferred embodiments, polymeric material can be molded through an injection molding process to the distal ends of the metal proximal portions 108-1, 108-2. In some examples, the polymeric material may be mixed with other materials such as glass particles or fibers to form a composite material that is stronger than the polymeric material alone or that has other desirable properties. The molded tips 114-1, 114-2 of the polymeric distal portions 108-1, 108-2 can be more precisely fabricated and at a lower cost than trying to form or machine similar structures with forceps made entirely of metal. As will be described in further detail below, the distal portions 110-1, 110-2 can be fabricated with various designs. For example, some embodiments of the tips 114-1, 114-2 have textured or serrated grasping platforms, and yet other embodiments of the tips 114-1, 114-2 have flat or hooked shapes.

In some examples, the injection molding process may be a two-step injection process. For example, a first injection process may be used to form a stiff core for the polymeric distal portions 110-1, 110-2. A second injection process may then be used to form a softer material such as silicone. The second injection process may form a coating on the core of the polymeric distal portions 110-1, 110-2.

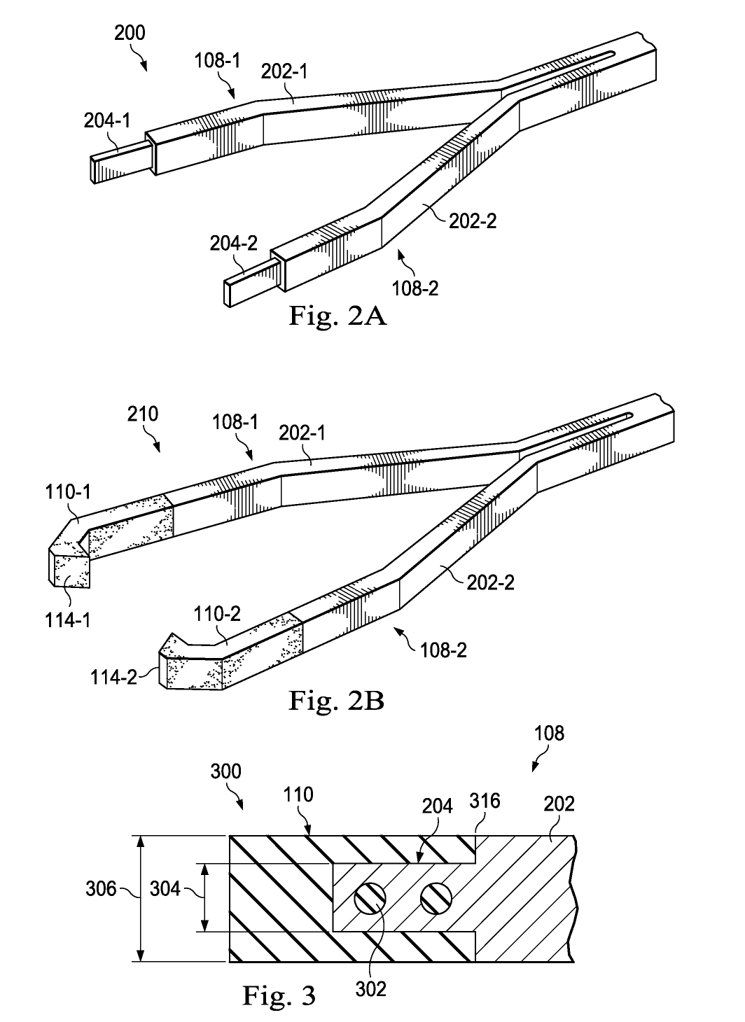

FIGS. 2A and 2B are diagrams showing a perspective view of a portion of the forceps before and after the polymeric portions are formed on the metal portions. FIG. 2A illustrates a metal skeleton 200 shaped to form the proximal portions (e.g. 108-1, 108-2, FIG. 1) before the polymeric portions are molded to the metal skeleton 200. FIG. 2B illustrates a portion of the forceps 210 after polymeric portions have been molded to the metal skeleton 200.

According to the present example, the metal skeleton 200 includes the proximal portions 108-1, 108-2. Each proximal portion 108-1, 108-2 includes a first sized portion 202-1, 202-2 and a second sized portion 204-1, 204-2. Specifically, proximal portion 108-1 includes first sized portion 202-1 and second sized portion 204-1. Likewise, proximal portion 108-2 includes first sized portion 202-2 and second sized portion 204-2.

The first sized portions 202-1, 202-2 form most of the proximal portions 108-1, 108-2. The first size portions 202-1, 202-2 are designed to provide strength and stiffness to the forceps. In one example, the first size portions 202-1, 202-2 have a substantially rectangular cross-sectional shape. Other cross-section shapes such as elliptical cross-sections or rounded rectangular cross-sections may be used as well.

The second size portions 204-1, 204-2 extend from the distal end of the first size portions 202-1, 202-2. The second size portions 204-1, 204-2 may have the same type of cross-sectional shape as the first sized portions 202-1, 202-2. Here, the second sized portions 204-1, 204-2 are smaller than the first size portions 202-1, 202-2 in at least one dimension. The second size portions 204-1, 204-2 may also be referred to as extensions. The second size portions 204-1, 204-2 are the parts of the metal skeleton 200 over which the polymeric distal portions 110-1, 110-2 are formed.

FIG. 2B illustrates polymeric distal portions 110-1, 110-2 that have been molded onto second sized portions 204-1, 204-2 forming the distal end of the metal skeleton 200. Specifically, the first polymeric distal portion 110-1 has been formed over the first second sized portion 204-1 and the second distal polymeric portion 110-2 has been formed over the other second sized portion 204-2. The polymeric distal portions 110-1, 110-2 correspond to the distal portions 110-1, 110-2 illustrated in FIGS. 1A and 1B. The polymeric portions 110-1, 110-2 include tips 114-1, 114-2 that correspond to the tips 114-1, 114-2 illustrated in FIGS. 1A and 1B.

FIG. 3 is a diagram showing a cross-sectional view of an interface 300 between a metal proximal portion 108 and a polymeric distal portion 110 of a forceps (e.g. 100, FIG. 1). According to the present example, the metal proximal portion 108 includes a first size portion 202 and a second sized portion 204 over which the polymeric distal portion 110 is formed. The first sized portion 202 has a first thickness 306 and the second sized portion 204 has a second, smaller thickness 304. The thicknesses 304, 306 may correspond to either height and/or width.

In one example, extension 204 includes structural features 302 designed to mechanically secure the polymeric distal portion 110 to the second sized portion 204 of the metal proximal portion 108 via interfering shapes or material, for example. In the example of FIG. 3, the features 302 are holes through which polymeric material can flow during the molding process that is used to create the polymeric distal portion 110. Other features such as grooves, dimples, notches, or serrated edges, for example, formed on the second sized portion 204 may be used as well to create a form closure.

According to the present example, the polymeric distal portion 110 has an outer surface shaped and sized to substantially match the shape and size of the first sized portion 202 of the metal proximal portion 304 to form a smooth transition from the polymeric distal portion 110 to the first sized portion 202. In other words, the thickness 306 of the polymeric distal portion 110 is the same as the thickness 306 of the metal portion 202. This creates a substantially continuous surface 316 at the outer interface between the polymeric portion 302 and the metal portion 304. In some examples, however, the polymeric portion 302 may have a different thickness than the metal portion 304 in either height or width.

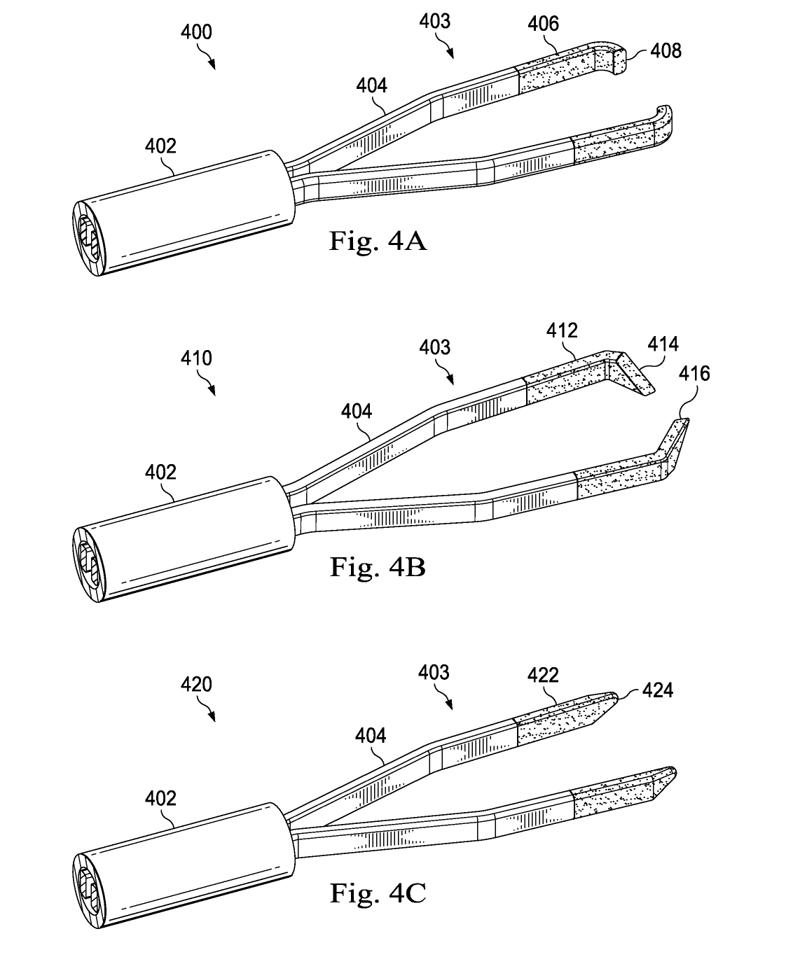

FIGS. 4A, 4B, and 4C are diagrams showing illustrative forceps with similar metal proximal portions and varying polymeric proximal portions. The metal skeletons (e.g. 200, FIG. 2) can be mass produced and similar metal skeletons can have varying polymeric distal portions formed thereon. FIG. 4A illustrates one example of a forceps 400. In this example, the forceps 400 includes a shaft 402 and arms 403 having a metal proximal portion 404 and a polymeric distal portion. The polymeric distal portion 406 includes hook shaped tips 408.

FIG. 4B illustrates another example of a forceps 410. This forceps 410 may have a shaft 402 and proximal portion 404 that is identical to the shaft 402 and proximal portion of the forceps 400 of FIG. 4A. But, forceps 410 has a different type of polymeric portion 412. Specifically, the forceps 410 has asymmetrical tips 414, 416. The first polymeric tip 414 is larger than the second polymeric tip 416. Such a design may provide various advantages for certain types of ophthalmic procedures.

FIG. 4C illustrates another example of a forceps 420. Like forceps 410, forceps 420 has a shaft 402 and proximal portion 404 that is identical to that of forceps 400. But, forceps 420 has a different proximal portion 422. Specifically, forceps 420 has flat shaped tips 424.

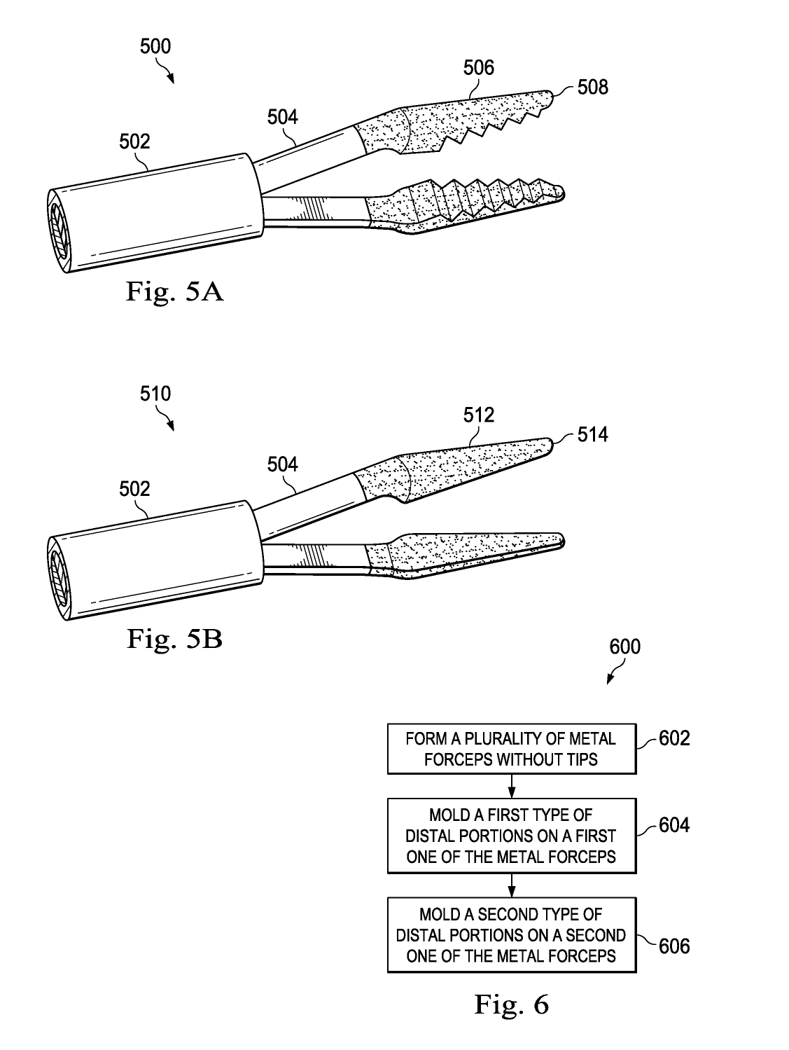

FIGS. 5A-5B are diagrams showing illustrative forceps with similar metal proximal portions and varying polymeric plastic portions. In one example, the shaft 502 and proximal metal portions 504 are identically shaped to the shaft 402 and proximal metal portions 404 of FIGS. 4A-4C. In some examples, however, the shaft 502 and proximal metal portions 504 may be differently shaped than the shaft 402 and proximal metal portions 404 of FIGS. 4A-4C.

FIG. 5A illustrates one example of a forceps 500. Forceps 500 has a shaft 502 and a proximal portion made of a metal material as well as a distal portion 506 made of a polymeric material. In the present example, the polymeric distal portion 506 includes serrated tips 508. The tips 508 also narrow in width as they extend further distally.

FIG. 5B illustrates another example of a forceps 510. Forceps 510 has a shaft 502 and a proximal portion 504 made of a metal material. The shaft 502 and proximal portion 504 may be identical in shape to the shaft 502 and proximal portion 504 of forceps 500 illustrated in FIG. 5A. The forceps 510 also includes a distal portion 512 made of a polymeric material. In the present example, the polymeric distal portion 512 includes flat tips 514. The tips 514 also narrow in width as they extend further distally.

FIG. 6 is a flowchart showing an illustrative method 600 of fabricating metal forceps with varying polymeric distal portions. According to the present example, at 602, a plurality of metal forceps are formed without tips. Such forceps may be referred to as metal skeletons. The metal skeletons (e.g. 200, FIG. 2) can be fabricated using standard metal fabrication methods such as laser cutting and wire Electrical Discharge Machining (EDM). The metal skeletons include two arms that are outwardly biased. In some examples, the arms come together and are joined to bodies that are small enough to fit within a shaft having a diameter of less than one millimeter. The distal ends of the arms include extensions that are smaller than the rest of the metal arms in at least one dimension.

At 604, a first type of distal portion may be molded on a first one of the metal forceps skeletons. The distal portions are molded over the extensions of the metal skeletons. As described above, those extensions may include features that create mechanical interference that allow the molded distal portions to better connect with the metal skeletons. In preferred techniques, an injection molding process may be used to form the first type of distal portions. However, other processes may be used to form the distal portions. Some embodiments of the distal portions may be designed for specific types of ophthalmic procedures. For example, as described above, the distal portions may be hook shaped, flat shaped, textured or serrated. Other shapes are contemplated as well. In some examples, additional material may be mixed with a polymeric material used in the injection molding process to form a composite material having desired properties. Such additional material may include glass pieces or fibers that may add additional strength to the polymeric material. In some examples, a metal injection molding process may be used to form the distal portions. In such cases, the distal portions are made of a different type of material than the proximal portions. For example, the distal portions may be made out of a metal that is better suited to an injection molding process.

At 606, a second type of distal portion may be molded on a second one of the metal forceps skeletons. Thus, while the first metal skeleton and second metal skeleton may be identical and come from the same manufacturing line, they may be molded with different types of distal portions. Again, an injection molding process may be used to form the second type of distal portions. The second type of distal portions may be different than the first type of distal portions. For example, if the first type of distal portions has flat edges, then the second type of distal portions may have serrated edges.

Through use of principles described herein, forceps can provide various structural advantages. Specifically, the metal proximal portions of the forceps provide a higher grasping force. Additionally, the molded polymeric distal portions can have various and intricate shapes that are difficult to form with a metal material. Furthermore, such forceps can be manufactured more efficiently. Specifically, metal skeletons for various forceps designs can be manufactured in bulk. Then, the varying tip designs of the distal portions can be molded onto those metal skeletons. Thus, only one metal fabrication process can be used instead of multiple metal fabrication processes for each tip design.

Persons of ordinary skill in the art will appreciate that the embodiments encompassed by the present disclosure are not limited to the particular exemplary embodiments described above. In that regard, although illustrative embodiments have been shown and described, a wide range of modification, change, and substitution is contemplated in the foregoing disclosure. It is understood that such variations may be made to the foregoing without departing from the scope of the present disclosure. Accordingly, it is appropriate that the appended claims be construed broadly and in a manner consistent with the present disclosure.

What is claimed is:

1. An ophthalmic surgical instrument comprising:
a shaft comprising a lumen having a longitudinal axis;
a first arm extending from the lumen, the first arm comprising:
a first proximal portion formed of a metal first material, the first proximal portion comprising a first shaft engaging portion that is angled away from the longitudinal axis of the shaft; and
a first distal portion attached onto the first proximal portion and having a first tip, the first distal portion formed of a second material different than the metal first material; and
a second arm extending from the lumen, the second arm comprising:
a second proximal portion formed of the metal first material, the second proximal portion comprising a second shaft engaging portion that is angled away from the longitudinal axis of the shaft; and
a second distal portion attached onto the second proximal portion and having a second tip, the second distal portion formed of the second material;
wherein the first and second shaft engaging portions are configured to engage the shaft such that movement of the shaft with respect to the first and second arms moves the first and second arms together such that the first tip moves towards the second tip in a manner permitting grasping of tissue.

2. The instrument of claim 1, wherein the second material comprises a polymeric material.

3. The instrument of claim 1, wherein the second material comprises a metal material that is suitable for an injection molding process.

4. The instrument of claim 1, wherein a distal end of the first proximal portion comprises a first sized portion and a second sized portion extending distally from the first sized portion, the second sized portion being smaller than the first sized portion.

5. The instrument of claim 4, wherein a proximal end of the first distal portion is molded over the second sized portion.

6. The instrument of claim 4, wherein the distal portion is similar in dimension to the first sized portion of first proximal portion.

7. The instrument of claim 4, wherein the second sized portion comprises a feature into which the polymeric material of the first distal portion is molded.

8. The instrument of claim 1, wherein the first tip comprises a first flat edge and the second tip comprises a second flat edge configured to engage with the first flat edge.

9. The instrument of claim 1, wherein the first tip comprises a first serrated edge and the second tip comprises a second serrated edge configured to engage with the first serrated edge.

10. The instrument of claim 1, wherein the first tip is formed to have a textured grasping platform.

11. The instrument of claim 1, wherein the first tip comprises a hook shape.

12. The instrument of claim 1, wherein the first and second distal portions comprise at least one of a fiber material or a glass material within the polymeric material

13. An ophthalmic instrument comprising:
a shaft comprising a lumen;
a first arm extending from the lumen, the first arm comprising:
a first proximal portion formed of a metal material, the first proximal portion comprising a first sized portion and a second sized portion extending distally from the first sized portion, the second sized portion being smaller in at least one dimension than the first sized portion; and
a first distal portion formed over the second sized portion of the first proximal portion, the first distal portion comprising a polymeric material; and
a second arm extending from the lumen, the second arm comprising:
a second proximal portion formed of the metal material, the second proximal portion comprising a first sized portion and a second sized portion extending distally from the first sized portion, the second sized portion being smaller in at least one dimension than the first sized portion; and
a second distal portion formed over the second sized portion of the second proximal portion, the second distal portion comprising the polymeric material.

14. The instrument of claim 13, wherein the first arm comprises a first shaft engaging portion that is angled away from a longitudinal axis of the shaft and the second arm comprises a second shaft engaging portion that is angled away from the longitudinal axis.

15. The instrument of claim 14, wherein the first and second shaft engaging portions are configured to engage the shaft such that movement of the shaft with respect to the first and second arms moves the first and second arms together such that a first tip of the first distal portion contacts a second tip of the second distal portion.

16. The instrument of claim 13, wherein a distal end of the first distal portion extends within a range of 1 to 3 millimeters from the shaft.

17. The instrument of claim 13, wherein a width of the first arm is within a range of 0.05 to 0.3 millimeters.

18. A method for fabricating ophthalmic instruments, the method comprising:
forming a plurality of metal forceps, each of the metal forceps comprising:
a first arm having a first extension that extends distally from the first arm and is smaller than the first arm along at least one dimension; and
a second arm having a second extension that extends distally from the second arm and is smaller than the second arm along at least one dimension;
molding a first type of polymeric tip over the first extension and the second extension of a first one of the plurality of metal forceps; and
molding a second type of polymeric tip over the first extension and the second extension of a second one of the plurality of metal forceps.

19. The method of claim 18, wherein molding the first type of polymeric tip and molding the second type of polymeric tip comprises an injection molding process.

20. The method of claim 18, wherein the first type of polymeric tip has a different shape than the second type of polymeric tip.
